(19) United States
(12) Patent Application Publication
(10) Pub. No.: US 2011/0196261 A1
(43) Pub. Date: Aug. 11, 2011

(54) LANCING DEVICE WITH IMPROVED GUIDANCE MECHANISM

(76) Inventors: Avi M. ROBBINS, Atlanta, GA (US); Gil KAN, Atlanta, GA (US); Brian M. COLLINS, Denver, CO (US); Christopher J. RUF, Marietta, GA (US)

(21) Appl. No.: 13/005,181

(22) Filed: Jan. 12, 2011

Related U.S. Application Data
(60) Provisional application No. 61/294,664, filed on Jan. 13, 2010.

(51) Int. Cl. A61B 5/151

(52) U.S. Cl. 600/583

(57) ABSTRACT

A lancing device having improved guidance features and a drive mechanism subassembly that is generically compatible with a variety of outer housing configurations, and an improved assembly method for fabricating a lancing device.

### CROSS-REFERENCE TO RELATED APPLICATION

This application claims the benefit of U.S. Provisional Patent Application Ser. No. 61/294,664, filed Jan. 13, 2010, the entirety of which is hereby incorporated herein by reference for all purposes.

### TECHNICAL FIELD

The present invention relates generally to the field of medical devices, and more particularly to a lancing device for blood sampling and testing, and to methods of assembly and use of lancing devices.

### BACKGROUND

Lancing devices are utilized for penetrating the skin of a human or animal subject at a lancing site to obtain a sample of blood or other body fluid for medical testing, as in blood-typing or blood-glucose testing. Known lancing devices commonly include a housing containing a drive mechanism, a charging mechanism for energizing the spring or other drive means of the drive mechanism, and a release mechanism for releasing the drive mechanism upon actuation.

A lancet is typically propelled by the drive mechanism from a retracted position within the housing to an extended position wherein a sharp tip portion of the lancet projects from the housing to prick the subject's skin at a desired lancing site. A depth-control mechanism can optionally be provided to adjust the penetration depth of the lancet.

The lancet is typically a disposable component that is removably mounted into a receiver or lancet carrier portion of the drive mechanism of a lancing device. A used lancet typically is removed from the lancet carrier after sampling for disposal. An ejection-mechanism can optionally be included for discharge of the used lancet from the lancing device. A new, sterile lancet is then replaced into the lancet carrier for further sampling.

It is to the provision of improved lancing devices and methods of assembly and use thereof that the present invention is primarily directed.

### SUMMARY

In example embodiments, the present invention provides a lancing device having improved guidance features. Further example embodiments of the invention provide improved methods of assembly and use of lancing devices.

In one aspect, the present invention relates to a lancing device including an outer housing, a subassembly inner housing mounted within the outer housing, a lancet carrier translationally mounted within the subassembly inner housing, and a drive spring engaged between the lancet carrier and the subassembly inner housing

In another aspect, the invention relates to a method of assembly of a lancing device. The method includes the steps of installing a drive spring onto a lancet carrier, inserting the lancet carrier into a subassembly inner housing with a distal arm portion of the lancet carrier extending through an opening in the back of the subassembly inner housing, installing a return spring over the distal arm portion of the lancet carrier, and engaging a spring retainer between opposed forks of the distal arm portion to form a drive mechanism subassembly.

In another aspect, the invention relates to a lancing device comprising a subassembly housing defining an internal chamber, an open proximal end, a rear panel opposite the proximal end and defining an opening therethrough, and a sidewall between the proximal end and the rear panel. The lancing device preferably further includes a lancet carrier having a receiver at a proximal end thereof for engaging a lancet, an elongate distal arm opposite the proximal end, and a medial portion between the proximal end and the distal arm, wherein the elongate distal arm extends through the opening in the proximal end of the subassembly housing. The lancing device preferably further includes a drive spring coupled between the medial portion of the lancet carrier and the rear panel of the subassembly housing, and a return spring mounted onto the elongate distal arm portion of the lancet carrier opposite the rear panel from the drive spring.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a lancing device according to an example embodiment of the present invention.

FIGS. 2a and 2b show the lancing device of FIG. 1 in an uncharged or initial state.

FIG. 3 shows the lancing device of FIG. 1 with its cap being removed.

FIGS. 4a-4c show installation of a removable lancet into the lancing device of FIG. 1.

FIG. 5 shows the lancing device of FIG. 1 with its cap replaced after installation of a lancet.

FIGS. 6a and 6b show the lancing device of FIG. 1 in an actuated or lancing state.

FIGS. 7a and 7b show internal views of the lancing device of FIG. 1 in an actuated or lancing state.

FIG. 8 is a cross-sectional view of the internal drive mechanism of the lancing device of FIG. 1.

FIG. 9 is a cross-sectional view of the trigger mechanism of the lancing device of FIG. 1.

FIG. 10 is a cross-sectional view of a spring retainer portion of the lancing device of FIG. 1.

FIGS. 11a and 11b are partial cutaway views of the lancing device of FIG. 1, showing handle operation and charging of the device.

FIGS. 12-16 show the lancing device of FIG. 1 with portions sequentially removed to show internal components thereof.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

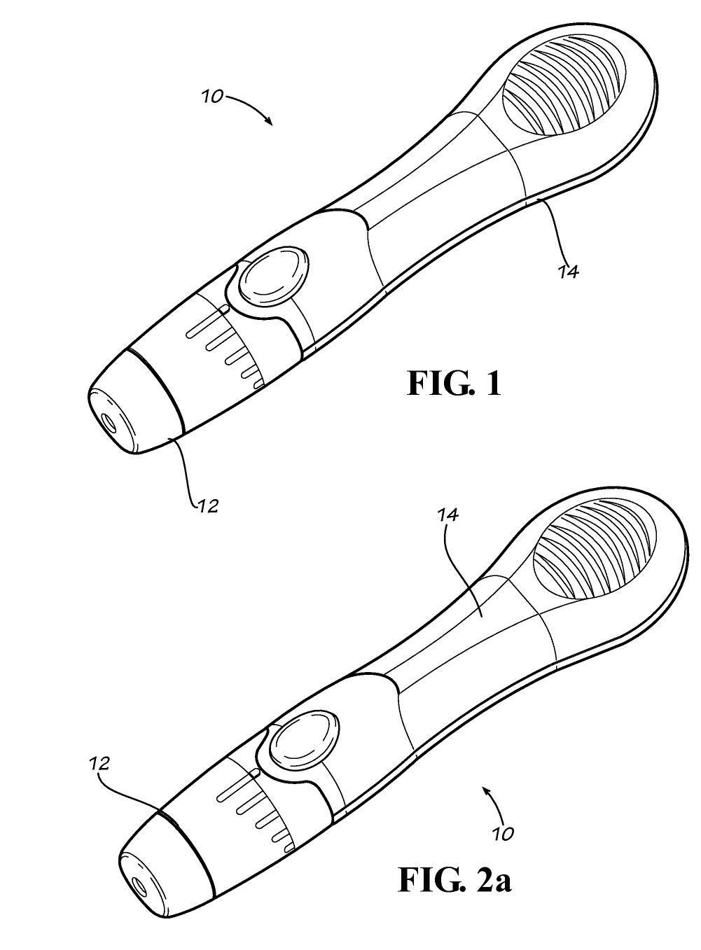

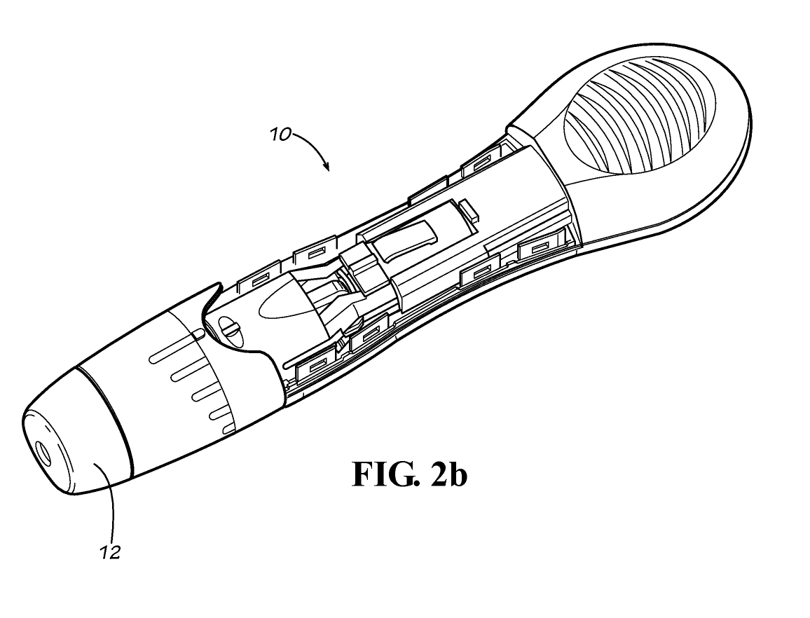

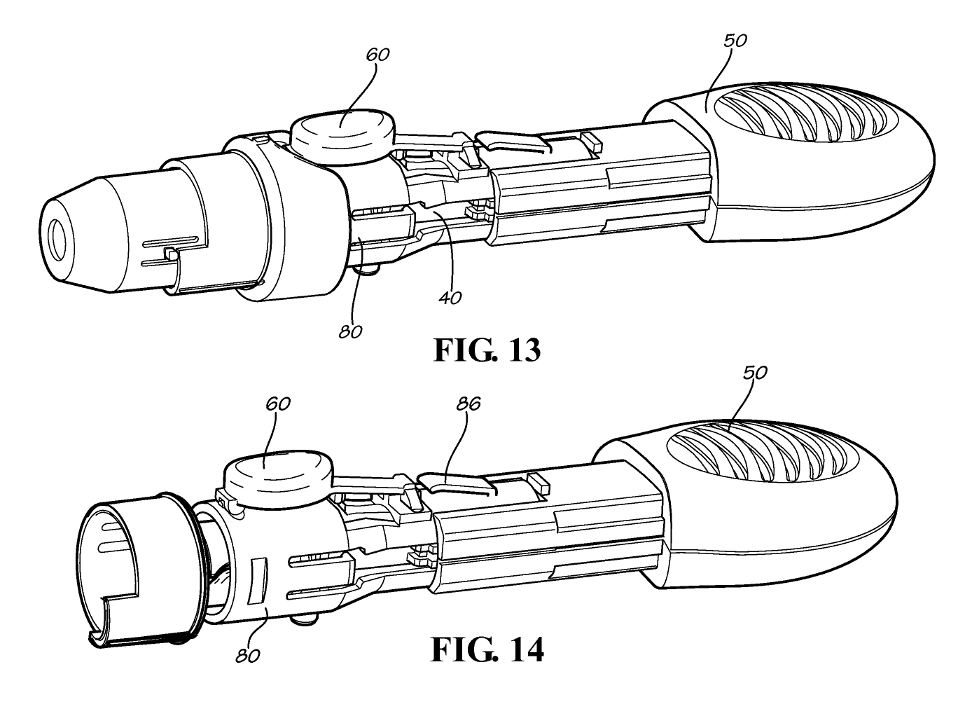

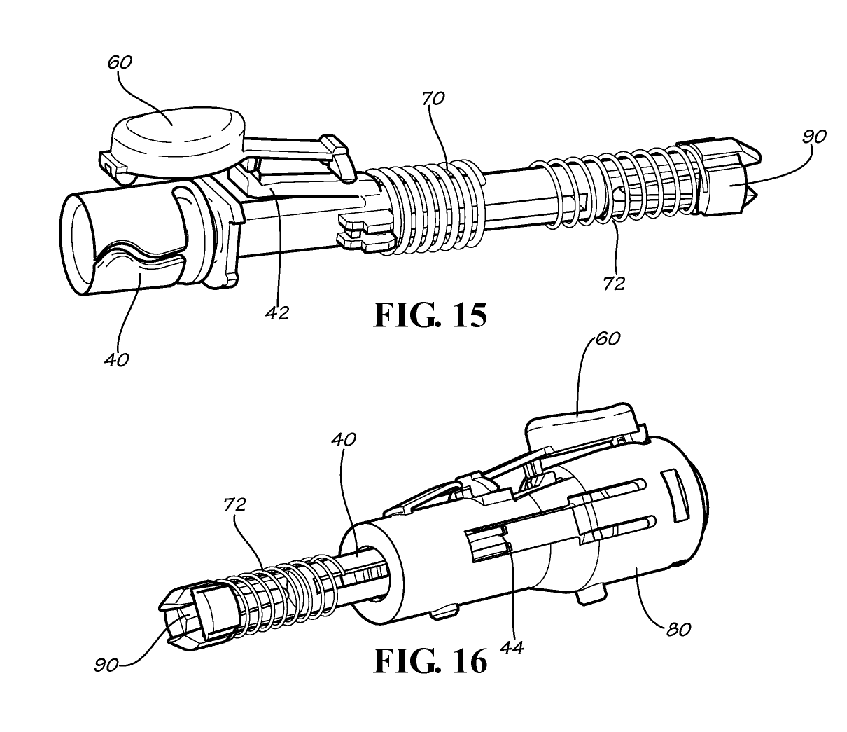

With reference now to the drawing figures, wherein like reference numbers represent corresponding parts throughout the several views, FIGS. 1-15 show various aspects and features of a lancing device 10 according to an example form of the invention. FIGS. 1 and 2 show the device 10 with its cap 12 attached to the housing 14 of the lancing device. FIG. 2b shows a portion of the housing removed to show the internal drive mechanism of the device in its uncharged state. The cap 12 optionally includes a depth-adjustment mechanism and depth setting indicia allowing the user to vary the penetration depth of the lancing procedure.

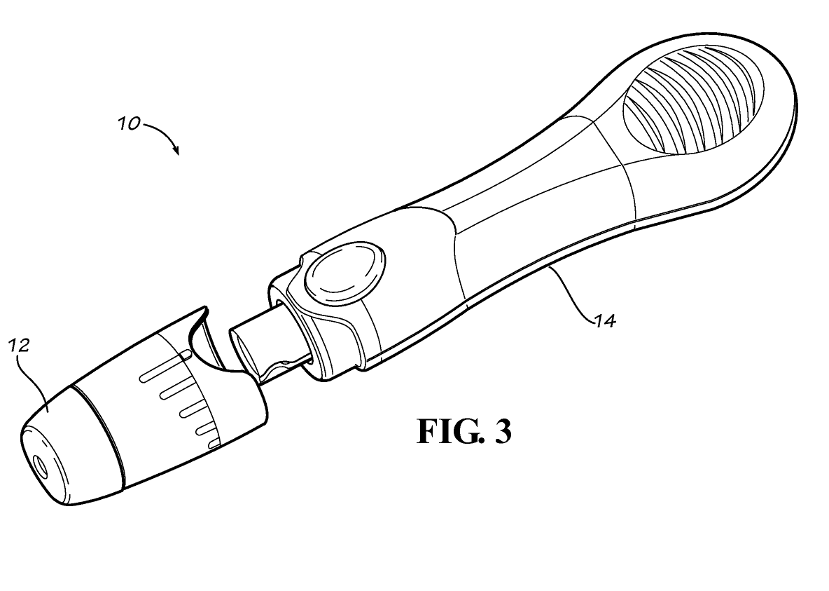

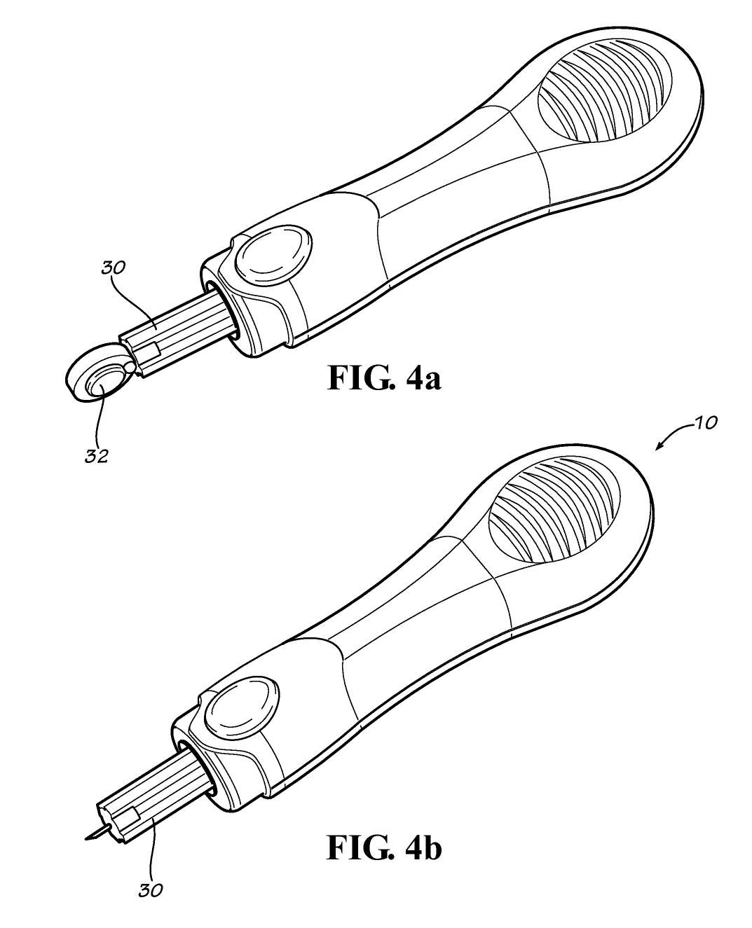

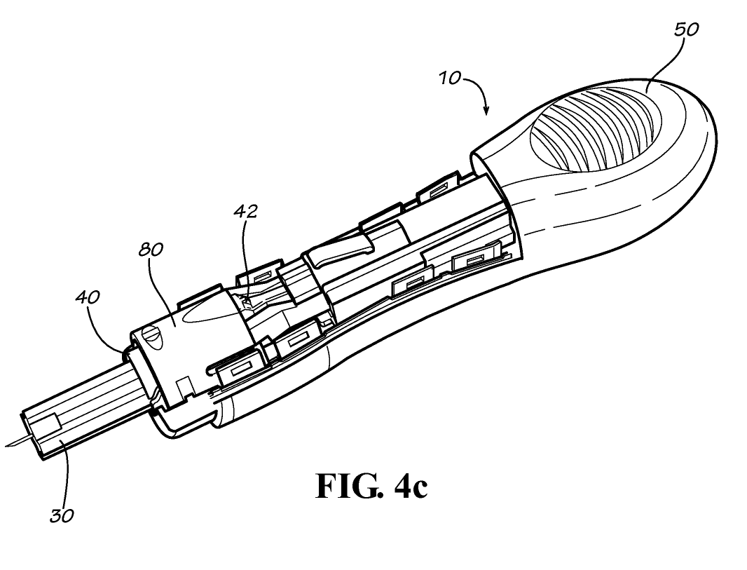

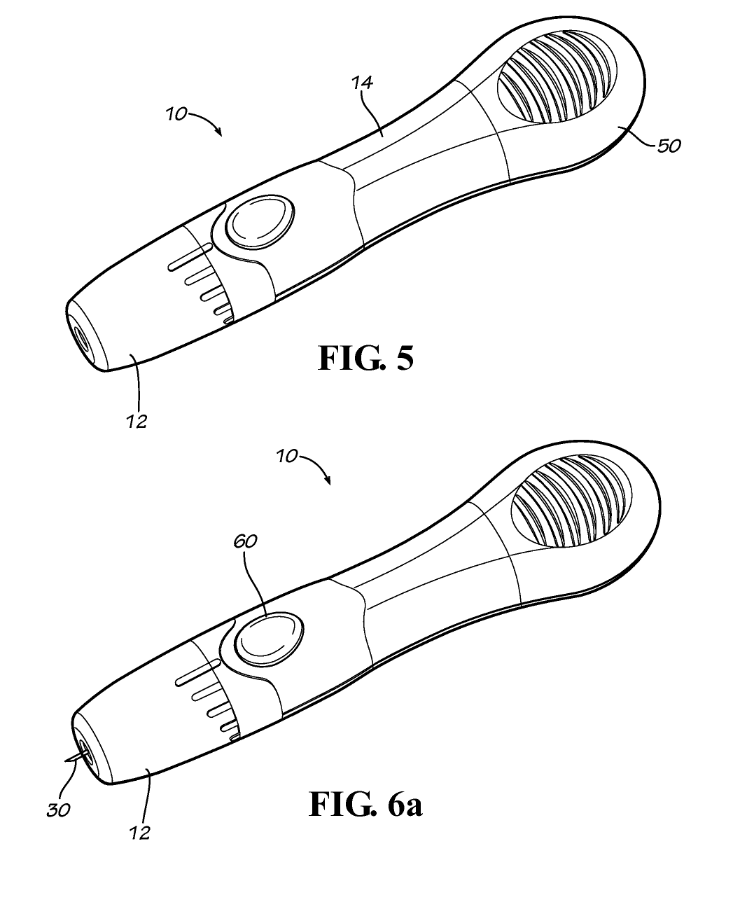

FIGS. 3-6 show a sequence of use of the lancing device 10, wherein the cap 12 is detached from the housing 14 (FIG. 3); allowing insertion of a lancet 30 into the drive mechanism, removal of the lancet's sterility tab 32 (FIG. 4); and then the cap 12 is replaced onto the body housing 14 (FIG. 5). After the lancet 30 is installed, the lancet holder or carrier 40 of the drive mechanism is retracted rearwards and a resilient cantilevered release finger 42 is raised to engage a cooperating trigger catch to place the device into a charged state. The lancet holder or carrier 40 of the drive mechanism may be retracted rearwards by pressing the lancet and/or carrier into the housing, or alternatively by retracting the charging handle 50 which is coupled to the lancet carrier.

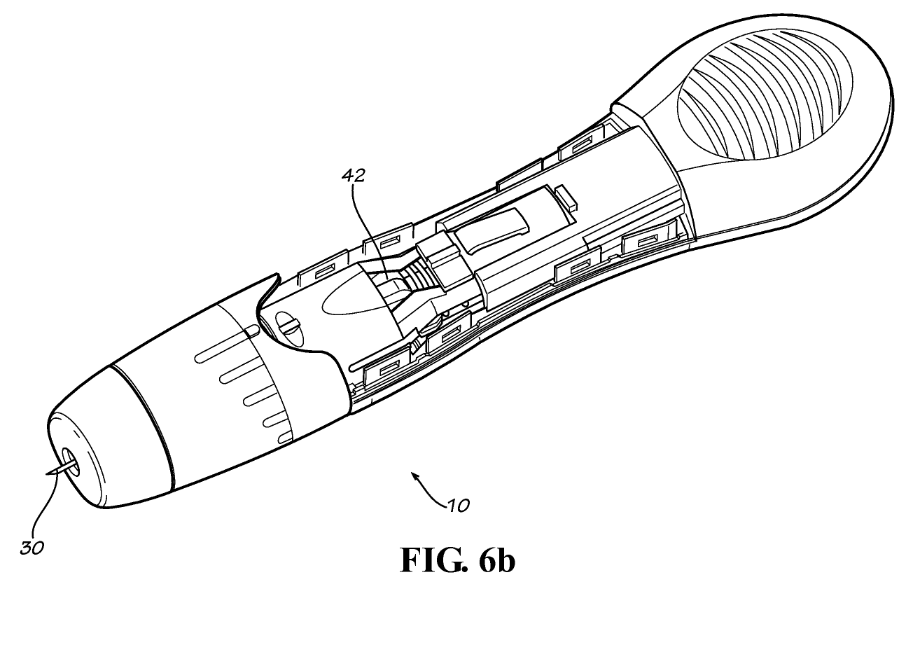

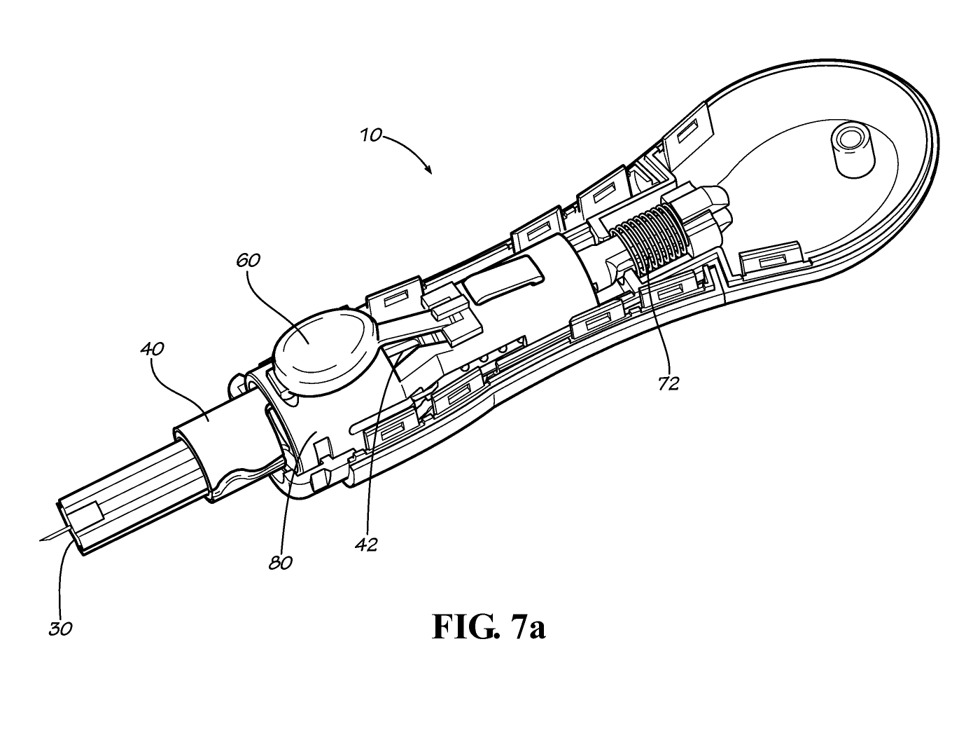

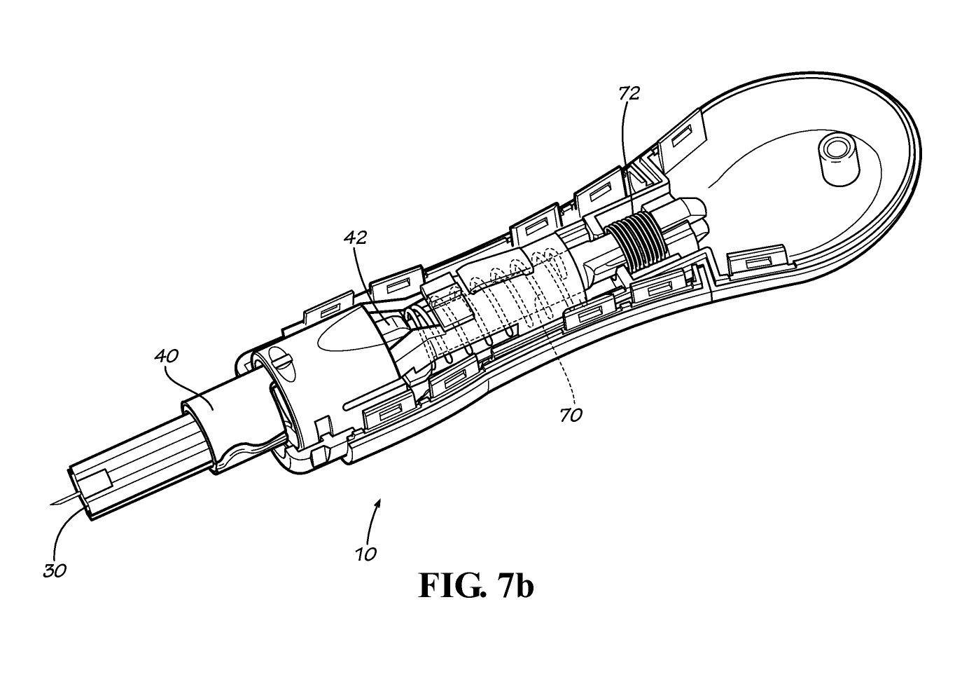

FIGS. 6 and 7 show actuation of the lancing device 10. The endcap 12 is placed against the subject's skin with its lancet opening at the intended lancing site. The release button 60 is pressed by the user, releasing the cantilevered release finger 42 from the cooperating trigger catch, thereby allowing the drive spring 70 to extend and propel the lancet carrier 40 forward until the sharp tip of the lancet 30 projects outwardly through the lancet opening to penetrate the subject's skin at the lancing site and generate a sample of blood for testing or other purpose. The return spring 72 compresses as the lancet carrier 40 traverses the forward portion of the lancing stroke, and then expands to retract the lancet carrier along a rearward portion of the lancing stroke, returning the lancet tip to a shielded position within the housing and endcap body.

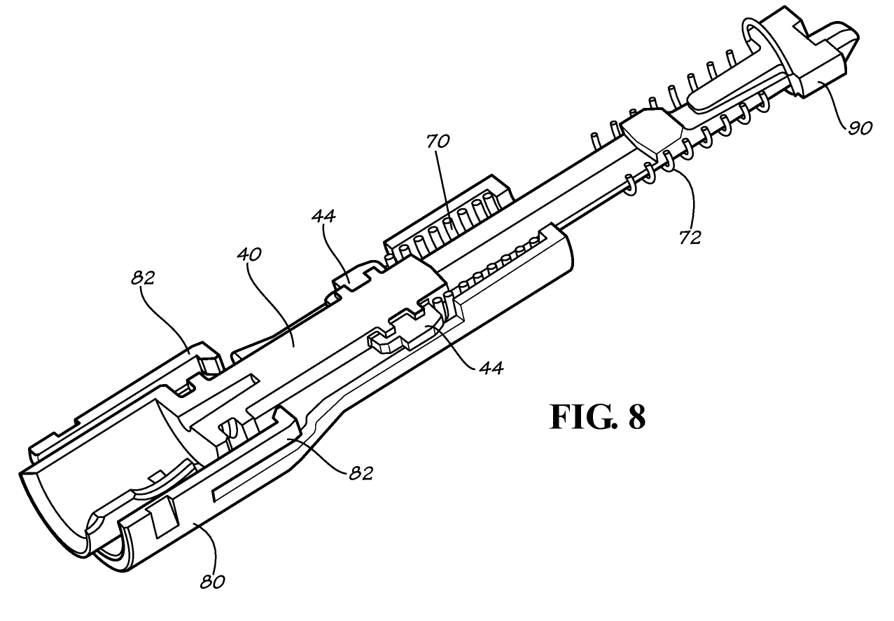

FIG. 8 shows greater detail of the drive mechanism of the lancing device, including a chassis or subassembly inner housing 80, within which the lancet carrier 40 is translationally mounted. The drive spring 70 is retained between an expanded portion of the lancet carrier 40 and a rear panel of the subassembly inner housing 80, biasing the lancet carrier proximally or forward. The return spring 72 is retained on a distal arm portion of the lancet carrier 40 by a spring retainer 90 (shown in greater detail in FIG. 10), which snaps into engagement between a pair of resilient forks on the lancet carrier.

Provision of the chassis or subassembly inner housing 80 allows assembly of the drive mechanism separate from the outer housing 14 by installation of the drive spring 70 onto the lancet carrier 40, insertion of the lancet carrier 40 into an internal chamber of the subassembly inner housing with the distal arm portion of the lancet carrier extending through an opening in the back of the subassembly inner housing, installation of the return spring 72 over the distal arm portion, and engagement of the spring retainer 90 thereon. The assembled drive mechanism may then be generically installed into any of a variety of outer housing configurations by mounting the chassis or subassembly inner housing 80 to one or more internal mounting components of the outer housing. The chassis or subassembly inner housing 80 is fixed in position within the outer housing 14 in the depicted embodiment, but optionally may be repositionable within the outer housing in alternate embodiments.

Wings 44 on the lancet holder 40 are installed past two cantilevers 82 of the subassembly inner housing 80, and once installed slide back and forth in lengthwise slots in the sidewalls of the subassembly housing to constrain the travel of the lancet holder to a linear axial path. The distally directed free ends of the cantilevers 82 act as a hard stop for abutment with the wings 44 to limit the stroke of the drive mechanism. This simplifies assembly and creates a hard stop with a degree of resilience that is not completely rigid. In this manner, the stop on the drive mechanism absorbs energy from the device, softening the impact sensed by the subject and potentially decreasing the degree of any pain perceived. This feature also adds guidance to the lancet holder as it traverses the lancing stroke, constraining the travel of the lancet tip to a linear axial path, also potentially decreasing the degree of any pain perceived.

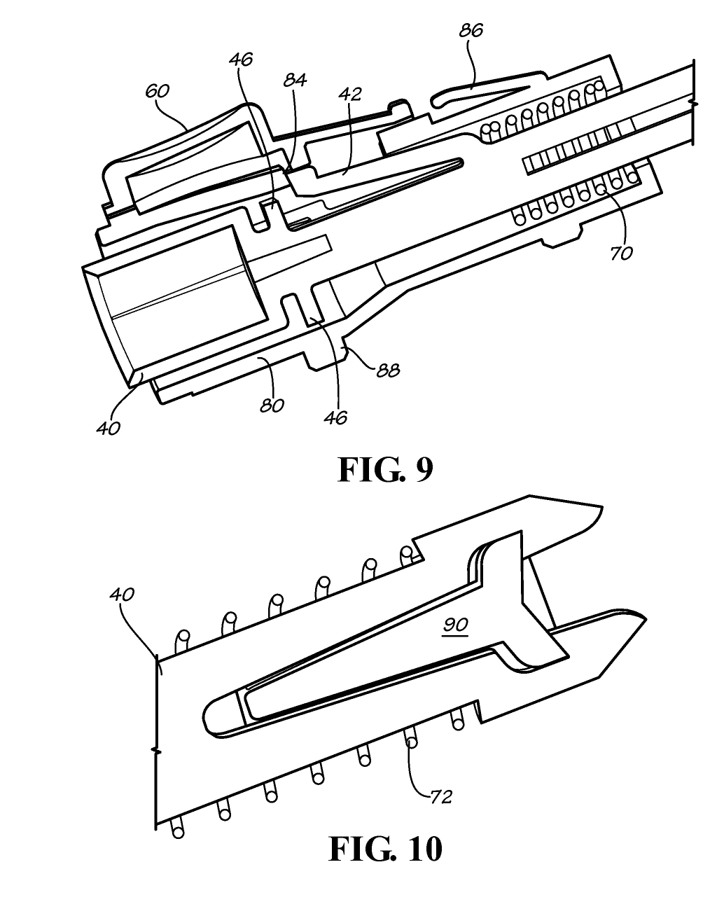

FIG. 9 shows the trigger mechanism and other portions of the drive mechanism of the lancing device in greater detail. The cantilevered release finger 42 engages the cooperating trigger catch portion 84 of the subassembly inner housing 80 to retain the drive mechanism in its charged state against the bias of the drive spring 70. When the trigger release button 60 is depressed, an actuation finger extending therefrom contacts the release finger 42 to release it from engagement with the trigger catch, actuating the device. A resilient cantilevered positioning arm 86 extends outwardly from the subassembly inner housing 80 to hold the drive mechanism housing tightly within the outer housing body 14, and to absorbs tolerances. A ring extension 46 extends outwardly from the lancet holder 40 for guidance, by fitting into the subassembly inner housing and keeping the system aligned. A projection or boss 88, and/or one or more additional connection members optionally project from the subassembly inner housing 80 for engagement with cooperating connection features of the outer housing 14.

FIG. 10 shows the spring retainer 90 and forked rear distal arm portion of the lancet carrier 40 of the lancing device in greater detail. The spring retainer acts as a lock to prevent the forks of the lancet holder from compressing, therefore preventing the return spring 72 from moving past a spring retention ledge on the lancet holder. The retainer 90 is snapped into position after the return spring 72 is assembled to the device. The drafts on the lancet holder and cantilevers are angled to provide the strongest possible retention and resist pull-out.

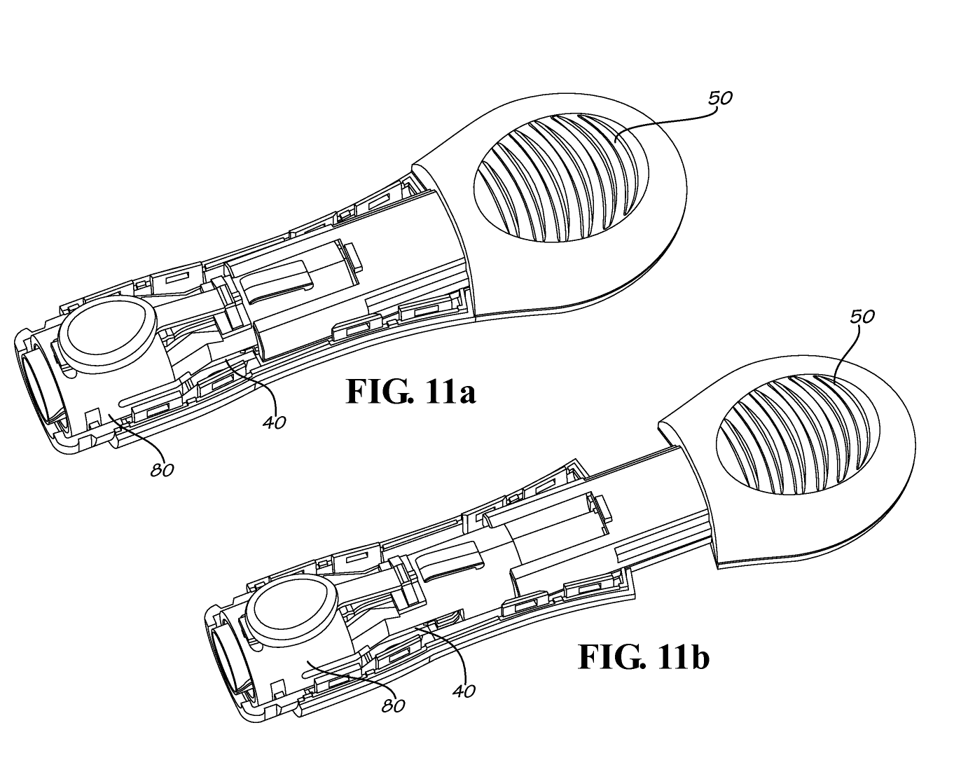

FIG. 11 shows operation of the charging handle 50 to charge the lancing device. The charging handle 50 is shaped so that they fit over the mechanism housing, adding guidance and providing rigidity to the handle while it is pulled outwards by the user.

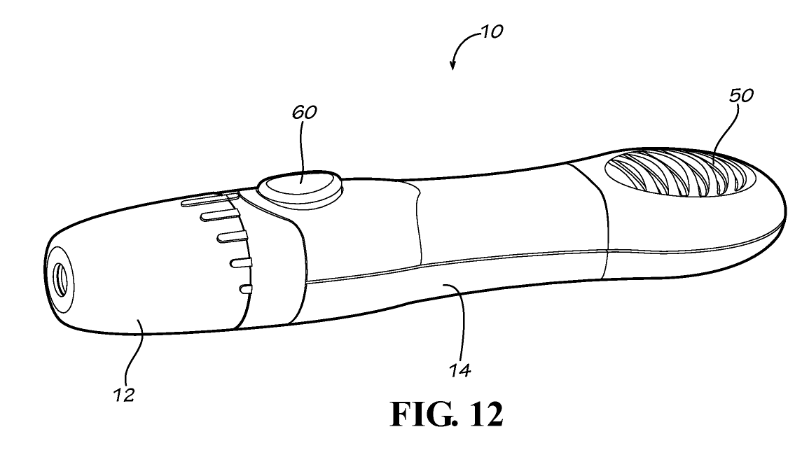

FIGS. 12-16 show additional views of the lancing device 10, with portions sequentially removed to show additional internal components thereof, as described above.

While the invention has been described with reference to preferred and example embodiments, it will be understood by those skilled in the art that a variety of modifications, additions and deletions are within the scope of the invention, as defined by the following claims.

What is claimed is:

1. A lancing device comprising an outer housing, a subassembly inner housing mounted within the outer housing, a lancet carrier translationally mounted within the subassembly inner housing, and a drive spring engaged between the lancet carrier and the subassembly inner housing.

2. The lancing device of claim 1, wherein the lancet carrier comprises a release finger for releasable engagement with a trigger catch portion of the subassembly inner housing to retain the lancet carrier in a retracted position with the drive spring compressed when the lancing device is charged.

3. The lancing device of claim 1, wherein the lancet carrier further comprises a forked rear extension arm having a return spring mounted thereon, and further comprising a spring retainer engaged between forks of the forked rear extension arm.

4. The lancing device of claim 1, wherein the subassembly inner housing further comprises a cantilevered positioning arm for positional contact with the outer housing body.

5. The lancing device of claim 1, wherein the lancet carrier comprises an alignment projection for positional guidance within the subassembly inner housing.

6. A method of assembly of a lancing device, the method comprising installing a drive spring onto a lancet carrier, inserting the lancet carrier into a subassembly inner housing with a distal arm portion of the lancet carrier extending through an opening in the back of the subassembly inner housing, installing a return spring over the distal arm portion of the lancet carrier, and engaging a spring retainer between opposed forks of the distal arm portion to form a drive mechanism subassembly.

7. The method of claim 6, further comprising installing the drive mechanism subassembly into an outer housing.

8. A lancing device comprising:
a subassembly housing defining an internal chamber, an open proximal end, a rear panel opposite the proximal end and defining an opening therethrough, and a sidewall between the proximal end and the rear panel;
a lancet carrier having a receiver at a proximal end thereof for engaging a lancet, an elongate distal arm opposite the proximal end, and a medial portion between the proximal end and the distal arm, wherein the elongate distal arm extends through the opening in the proximal end of the subassembly housing;
a drive spring coupled between the medial portion of the lancet carrier and the rear panel of the subassembly housing; and
a return spring mounted onto the elongate distal arm portion of the lancet carrier opposite the rear panel from the drive spring.

9. The lancing device of claim 8, wherein the sidewall of the subassembly housing has at least one lengthwise slot formed therein.

10. The lancing device of claim 9, wherein the sidewall of the subassembly housing further comprises a cantilevered finger having its free end at a proximal end of each lengthwise slot.

11. The lancing device of claim 9, wherein the lancet carrier comprises at least one wing projecting transversely outward from its medial portion, and sliding proximally and distally an associated one of the lengthwise slots in the sidewall of the subassembly housing to constrain the lancet carrier to a substantially linear lancing stroke.

12. The lancing device of claim 8, further comprising an outer body housing, the subassembly housing being at least partially mounted within the outer body housing.

13. The lancing device of claim 12, wherein the subassembly housing has a flexible cantilevered positioning arm extending outwardly therefrom, with its free end contacting the outer body housing to maintain alignment of the subassembly housing with the outer body housing.

14. The lancing device of claim 12, wherein the subassembly housing has a boss projecting therefrom for engagement with a cooperating connection feature of the outer body housing.

15. The lancing device of claim 12, further comprising an endcap detachably coupled to a proximal end of the outer body housing.

16. The lancing device of claim 13, wherein the endcap comprises an inner member and an outer member, the outer member positionally coupled to the inner member to adjust a lancing depth provided by the lancing device.

17. The lancing device of claim 12, further comprising a charging handle coupled to the lancet carrier to energize the drive spring.

18. The lancing device of claim 8, wherein the lancet carrier comprises a ring extension extending outwardly therefrom for sliding contact along an inner surface of the interior chamber of the subassembly housing to provide guidance of the lancet carrier along a lancing stroke.

19. The lancing device of claim 8, wherein the lancet carrier comprises a flexible release finger extending therefrom for releasable engagement with a cooperating trigger catch portion of the subassembly housing to retain the lancet carrier in a retracted position against the bias of the drive spring.

20. The lancing device of claim 19, further comprising a trigger release button for contacting the release finger to release it from engagement with the trigger catch, actuating the lancing device and releasing the lancet carrier to move under the bias of the drive spring from the retracted position to an extended position wherein a sharp lancet tip projects into a lancing position.

21. The lancing device of claim 8, wherein the elongate distal arm of the lancet carrier comprises a forked distal end having an interior receiver and an exterior shoulder for retaining the return spring.

22. The lancing device of claim 21, further comprising a spring retainer for engagement within the interior receiver of the forked distal end of the lancet carrier.
